(19) United States
(12) Patent Application Publication
(10) Pub. No.: US 2013/0345921 A1
(43) Pub. Date: Dec. 26, 2013

(54) PHYSIOLOGICAL MONITORING OF MOVING VEHICLE OPERATORS

(71) Applicant: Masimo Corporation, Irvine, CA (US)

(72) Inventors: Ammar Al-Ali, San Juan Capistrano, CA (US); Mohamed K. Diab, Ladera Ranch, CA (US); Massi Joe E. Kiani, Laguna Niguel, CA (US); Nikolai Marinow, Herrenberg (DE)

(21) Appl. No.: 13/923,888

(22) Filed: Jun. 21, 2013

Related U.S. Application Data
(60) Provisional application No. 61/663,425, filed on Jun. 22, 2012.

(51) Int. Cl. G05D 1/00; A61B 5/00; B60Q 3/00

(52) U.S. Cl. CPC G05D 1/0055; B60Q 3/00; A61B 5/0064
USPC 701/23; 297/217.6; 600/479; 600/476; 600/323

(57) ABSTRACT

The present disclosure relates to determining an physical state of a moving vehicle operator. In an embodiment, if it is determined that a vehicle operator is impaired, the vehicle is programed to automatically and safely stop a vehicle before an accident occurs. In an embodiment physiological sensors in the seat, steering wheel, or wireless sensors placed on the vehicle operator's body are used to determine an impairment state of a vehicle operator.

### CROSS-REFERENCE TO RELATED APPLICATIONS

This application claims a priority benefit under 35 U.S.C. § 119(e) to U.S. Provisional Application No. 61/663,425, filed Jun. 22, 2012, entitled “PHYSIOLOGICAL MONITORING OF MOVING VEHICLE OPERATORS,” which is hereby incorporated by reference in its entirety.

### FIELD

The present disclosure is related to the field of physiological monitoring.

### BACKGROUND

Moving vehicle accidents are a major source of property damage, personal injury and loss of life. Vehicle manufacturers have integrated numerous technologies into vehicles in an attempt to decrease injury or loss of life in the event of an accident. However, vehicle manufacturers have not found appropriate ways to automatically prevent accidents before they happen.

### SUMMARY

The present disclosure relates to determining an physical state of a moving vehicle operator. In an embodiment, if it is determined that a vehicle operator is impaired, the vehicle is programed to automatically and safely stop a vehicle before an accident occurs. In an embodiment physiological sensors in the seat, steering wheel, or wireless sensors placed on the vehicle operator's body are used to determine an impairment state of a vehicle operator.

### BRIEF DESCRIPTION OF THE DRAWINGS

FIG. 1 is an embodiment of physiological sensors placed in the seat bottom of a vehicle.

FIG. 2 is an embodiment of physiological sensors placed in the seat back of a vehicle.

FIGS. 3-5 illustrate embodiments of physiological sensors placed in a steering wheel of a vehicle.

FIG. 6 is an embodiment of a hat based sensor worn by a vehicle operator.

FIG. 7 is an embodiment of a glove based sensor worn by a vehicle operator.

### DETAILED DESCRIPTION

The present disclosure provides examples of physiological sensors incorporated into a vehicle and used to determine a physical state of an operator. The vehicle can be an automobile, a truck, a train, a plane, a boat, a submarine, a tractor or other construction equipment or any other moving vehicle. The physiological information can be used to determine whether the operator is experiencing a medical condition that may impair the operator's ability to control the vehicle. For example, this can include whether the operator is drowsy, experiencing a heart attack, a seizure or other medical ailment. In various embodiments, different parameters can be obtained in order to determine the state of the operator. For example, useful parameters for determining the physiological state of a vehicle operator include pulse rate, plethysmograph, arrhythmias or other heart conditions, oxygen saturation, respiration rate, ECG, temperature, carboxyhemoglobin, methemoglobin, total hemoglobin, glucose, consciousness, etc.

Once the physiological parameters are obtained, the vehicle can process the data to determine if the operator is experiencing a condition that places the vehicle at risk. The vehicle can be configured to automatically and safely stop itself in the event that the vehicle determines that the operator is unable to physically control the vehicle. In an airplane embodiment, the vehicle can switch to an autopilot feature. In an automobile, truck, train, boat or other land or sea based vehicle, the vehicle can slow itself down to a stop and turn on warning lights, such as hazard lights.

In an embodiment, when the vehicle determines an operator may be experiencing an impaired condition, the vehicle can request the driver to provide some input to verify that the driver is impaired. For example, the vehicle can ask the operator to audibly state whether the operator believes they are capable of operating the vehicle. In an embodiment, if the operator does not respond, the vehicle will automatically take control of itself. In an embodiment, the operator will be required to push an override button or provide some other task which verifies the operator is not impaired.

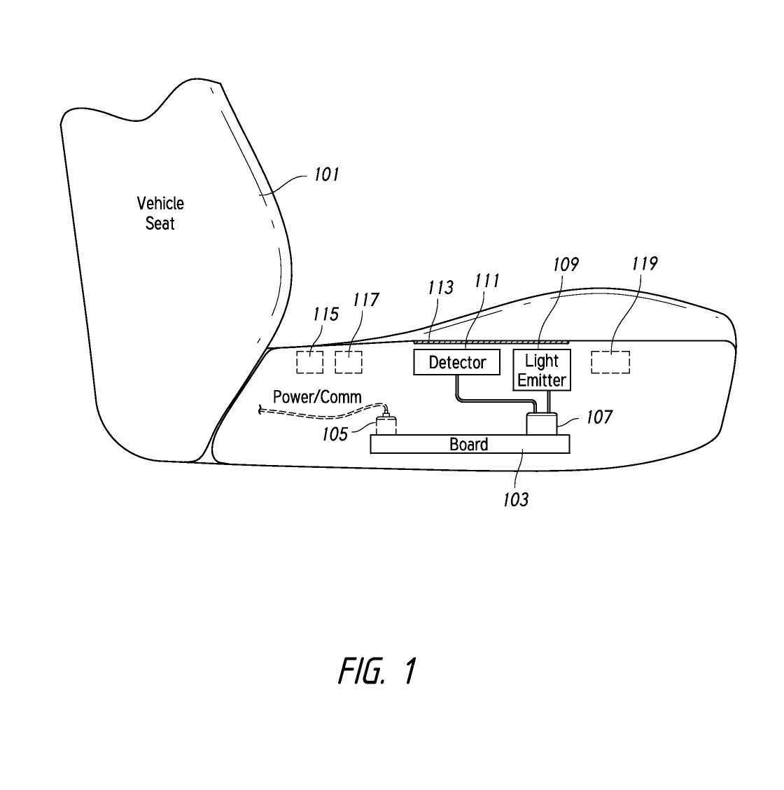

FIG. 1 illustrates an embodiment of a vehicle seat 101 incorporating sensors in the bottom of the seat. Seat 101 includes processor board 103. The processor board 103 receives power and communication over power/com lines 105. The processor board 103 also communicates with light emitter 109 and detector 111. Light emitter 109 and detector 111 are located beneath a fabric layer 113 out of sight of the vehicle operator. The light emitter 109 and detector 111 are located such that the light emitter shines light into the vehicle operators body and detector 111 detects the light as it reflects back. In an embodiment, the light emitter is located under the left thigh or buttocks of the vehicle operator. The left thigh is advantageous because generally the right leg is used for control of most automatic vehicles and thus the left thigh is left relatively motionless. The light emitter 109 is configured to be a deep tissue emitter, for example, by emitting a strong infrared signal. For example, the light emitter can be in the range of 100 mW to 1W. An infrared light can be used, as opposed to a red light, for example, so that it is invisible to the operator and will not distract the operator. Alternatively a red light, or combination of red and infrared light can be used. Other visible or non-visible lights can also be used as would be understood by a person of skill in the art from the present disclosure. In an embodiment, a pressure sensor senses that a vehicle operator is sitting on the emitter, triggering activation of the emitter.

FIG. 1 also illustrates optional sensors 115, 117 and 119. Optional sensors 115, 117 and 119 can be, for example, acoustic sensors or motion sensors. For example, the sensors can be made from piezoelectric film, an accelerometer, or any other type of motion or acoustic sensing system. Theses sensors can be used to pick up physiological signals such as pulse rate, respiration rate, breathing noises, etc. The physiological signals can filtered out from other irrelevant motion and/or acoustic signals attributable to movement of the vehicle or the operator.

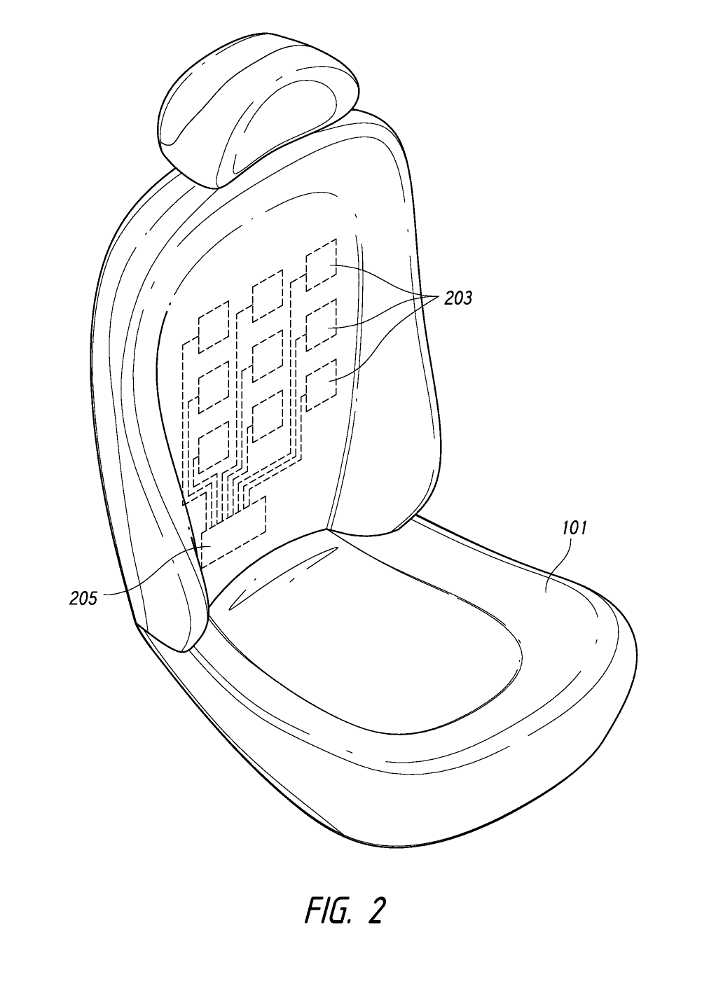

FIG. 2 illustrates an embodiment in which sensors are placed in the seat back of the vehicle. Sensors 203 can be acoustic, motion, or optical sensors as described above with respect to FIG. 1. The sensors can be placed in an array as illustrated or a single sensor can be used. An array of sensors can provide more information than can be gleamed from a single sensor. The sensors 203 can be electrically connected to the circuit board 205 as illustrated. In FIG. 2. The sensors 203 can determine, for example, heart movement, the strength of the heart, pulse rate and respiration rate.

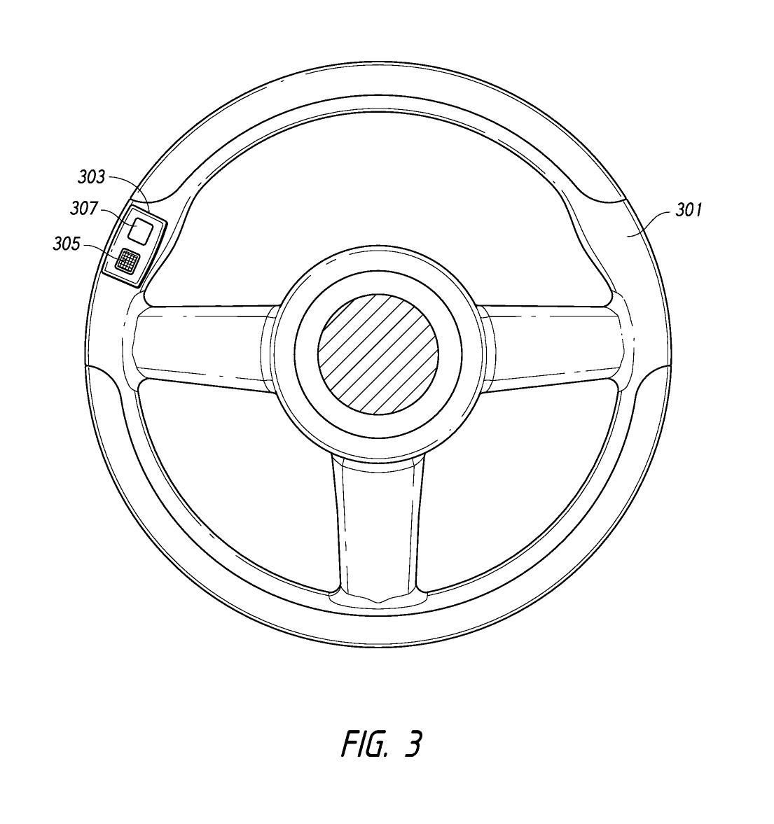

FIG. 3 illustrates an embodiment of a steering wheel 301 with an integrated optical reflectance sensor. The reflectance sensor includes an emitter 305, such as, for example, an infrared emitter and a detector 307. The reflectance center is located on a portion of the steering wheel 303 which is most likely to be held by the operator. In some embodiments, the location of the reflectance sensor is well marked so that the driver knows where to place is hands. In an embodiment, the reflectance sensor is located so that emitted light is emitted away from a driver, such as, for example, being emitted perpendicular to the driver or toward the road way so as not to distract a driver.

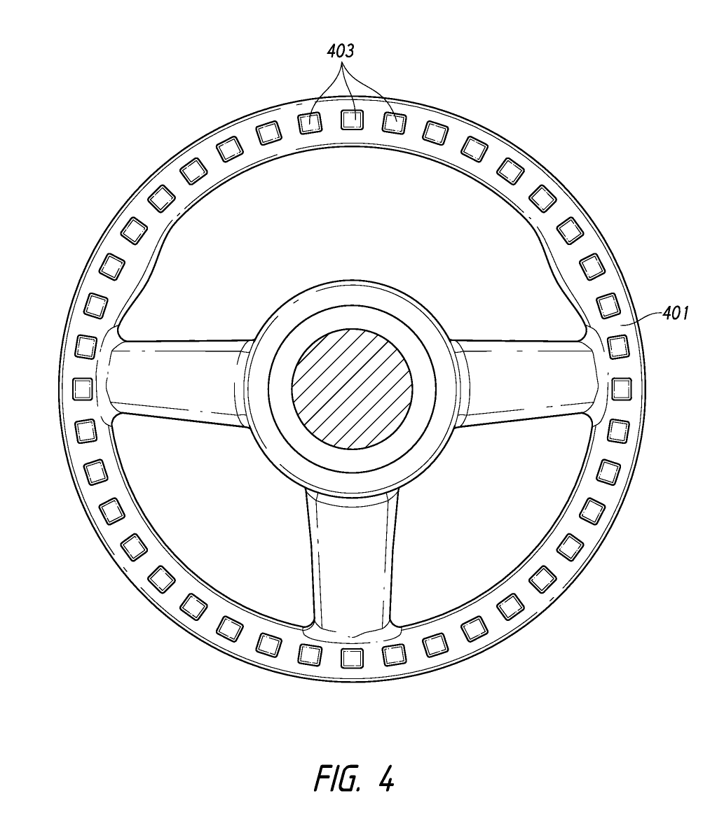

FIG. 4 illustrates another embodiment of a steering wheel 401 that includes sensors placed at various points around the circumference of the steering wheel 401. In this embodiment, multiple sensors 403, such as optical reflectance sensors, are placed around the wheel such that no matter where the operator places his hands, at least one sensor will detect the operator's physiological parameters. Again, an embodiment, the sensors are placed in a configuration that prevents emitted light from shining directly into a vehicle operators face to prevent distraction.

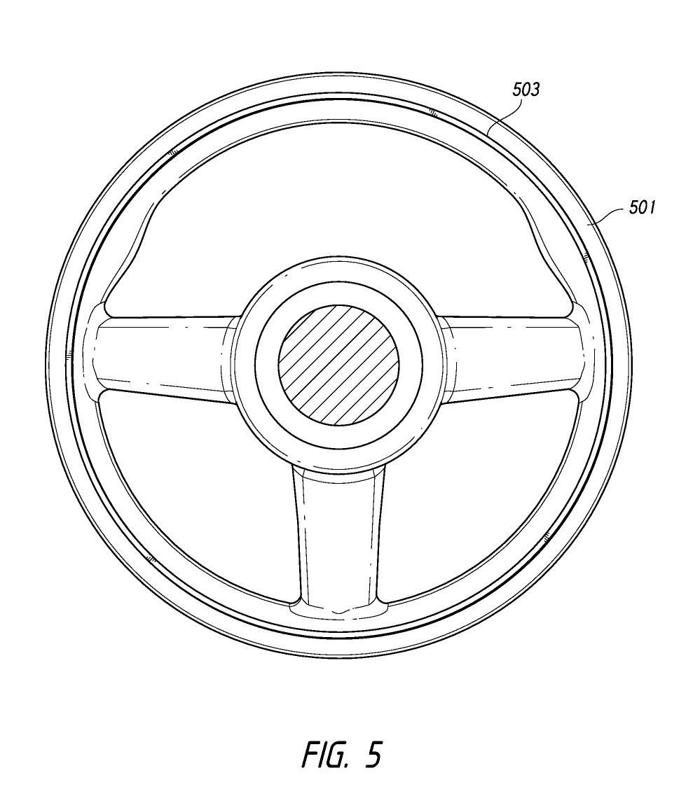

FIG. 5. illustrates another embodiment of a steering wheel 501 with integrated light piping sensor 503. The light piping sensor 503 acts as an optical reflectance sensor. Light is piped around the steering wheel. When a driver place his hands on the steering wheel, the light will be attenuated. The attenuated light is detected and processed to determine optical physiological measurements.

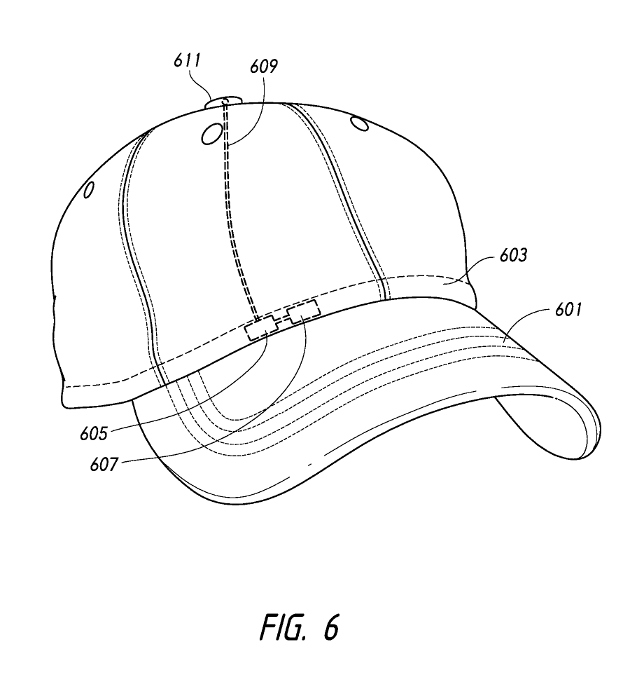

FIG. 6 illustrates an embodiment of a sensor integrated into a hat 601. The hat can be worn by the operator and can communicate wirelessly with processors in the vehicle. The hat 601 has a band 603 which places sensors 605, 607 against the forehead of the operator. The sensors communicate with transceiver 611 which processes and transmits data to the vehicle for further processing and/or display. The sensors 605, 607 can be an EEG sensor used to determine consciousness of the patient. The sensors 605, 607 can also be a reflectance based oximetry sensor, acoustic sensor, accelerometer sensor or the like.

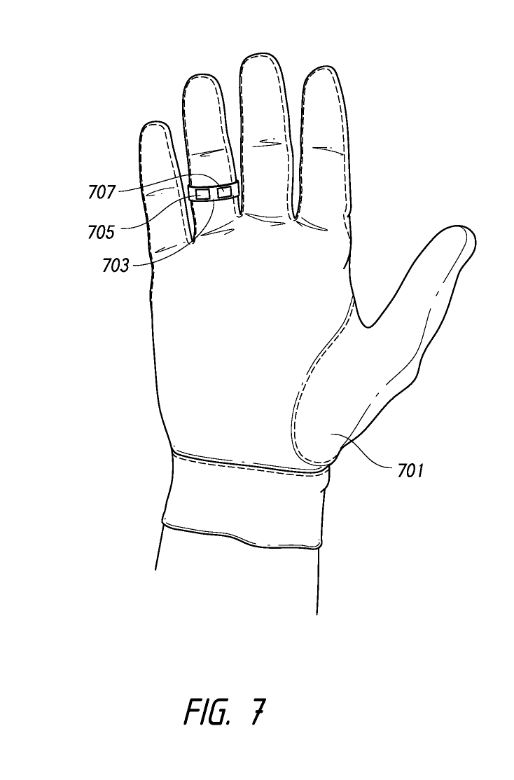

FIG. 7 illustrates a driving glove 701 with an integrated ring sensor 703. The ring sensor has light emitters 705 and a detector 707. The ring sensor can be a reflectance based sensor or a transmission based sensor. The sensor 703 can communicate wirelessly with the vehicle.

In another embodiment, an optical sensor is integrated into a Bluetooth device and placed on the operators ear. The Bluetooth device pairs with the vehicle and shares physiological information with the vehicle. In an embodiment, an optical ear sensor is integrated into the seat and is retractable, allowing the operatory to place the ear sensor on the ear while operating the vehicle.

In an embodiment, various physiological sensors are integrated into a watch, band or other wearable object. The watch or band can be recharged in a dedicated recharging station in the vehicle. In an embodiment, the watch or band can also include a wireless key that allows entry in the vehicle.

In an embodiment, an infrared laser can be configured to shine on the operator's face from a distance so as not to distract the vehicle operator. A camera is used to determine if the operator's skin color changes or whether the operator begins to sweat profusely. Sudden changes in an operator's condition can indicate an imminent threat.

In an embodiment, body penetrating radar can be used to measuring heart and lung movement. The radar can be used to extract information on changes in the operators physiology.

The sensor embodiments disclosed herein can be used in conjuction with known monitoring techniques, such as, for example, a pulse oximeter or acoustic monitoring device, both of which are commercially available from Masimo Corporation of Irvine, Calif.

What is claimed is:

1. A vehicle operator physiological monitoring system comprising:
a physiological monitor configured to measure a physiological state of a vehicle operator;
a processor in communication with the physiological monitor that determines if the operator is impaired and automatically takes control of the vehicle to prevent an accident.

2. The system of claim 1, wherein the physiological sensor is located in an operator's seat.

3. The system of claim 1, wherein the physiological sensor is located in a steering wheel.

4. The system of claim 1, wherein the physiological sensor is located in a hat worn by the operator.

5. The system of claim 1, wherein the physiological sensor is located a glove worn by the operator.

6. The system of claim 1, wherein the physiological sensor is located in a Bluetooth device worn by the operator.

7. A system configured to monitor a physiological state of a vehicle operator, the system comprising:
a vehicle operator seat;
at least one light emitter; and
at least one light detector, wherein the at least one light emitter and the at least one light detector are housed and form part of the vehicle operator seat.

8. The system of claim 7, wherein the at least one light emitter is configured to be shine light into the leg or buttocks of a vehicle operator;

9. The system of claim 8, wherein the at least one light emitter and the at leaste one light detector are placed below at least a first layer of fabric of the vehicle operator seat.

10. A method of determining a physiological state of a vehicle operator, the method comprising:
shining a light into a body portion of a vehicle operator, wherein the light is of sufficient power to penetrate clothing worn by the vehicle operator;
detecting light attenuated by the body portion of the vehicle operator;
processing the detected attenuated light to determine a physiological state of the vehicle operator.

11. The method of claim 10, wherein the physiological state is a pulse rate.

12. The method of claim 10, wherein the physiological state is oxygen saturation.

13. The method of claim 10, wherein the physiological state is respiration rate.

14. The method of claim 10, wherein the physiological state is an alertness level.
